(19) United States
(12) Plant Patent Application Publication
(10) Pub. No.: US 2007/0107105 P1
(43) Pub. Date: May 10, 2007

(54) Strawberry plant named 'Driscoll Sausalito'

(76) Inventors: Bruce D. Mowrey, Watsonville, CA (US); Larry T. Kodama, Watsonville, CA (US); JoAnne Coss, Salinas, CA (US); Michael Ferguson, Moorpark, CA (US)

Correspondence Address: JONES DAY, 222 EAST 41ST ST, NEW YORK, NY, 10017, US

(21) Appl. No.: 11/271,762

(22) Filed: Nov. 10, 2005

(51) Int. Cl. A01H 5/00

(52) U.S. Cl. PLT/208

(57) ABSTRACT

This invention relates to a new and distinct variety of strawberry named ‘Driscoll Sausalito’. The variety is similar to the varieties ‘Driscoll Lanai’ and ‘San Juan’. The variety is distinguished from ‘Driscoll Lanai’ and ‘San Juan,’ in particular, by its sweetness, acidity, internal color, longer petal length, longer flower diameter, anythocyanin color and coloration and moderate resistance to powdery mildew.

### LATIN NAME OF THE GENUS AND SPECIES OF THE PLANT CLAIMED

The variety is botanically identified as Fragaria×ananassa.

### VARIETY DENOMINATION

The strawberry variety denomination is ‘Driscoll Sausalito’.

### BACKGROUND OF THE INVENTION

The new variety originated as a result of a controlled cross between the strawberry plants ‘San Juan’ (U.S. Plant Pat. No. 12,899) and 14C185 (Unpatented Driscoll Variety) in an ongoing breeding program, and was discovered in Monterey County, California in 2000. The original seedling of the new cultivar was asexually propagated by stolons in a Nursery in Shasta County, California. Propagules were transplanted to a controlled breeding plot in Monterey County, California, where the variety was identified and selected for further evaluation. ‘Driscoll Sausalito’ was subsequently asexually propagated and underwent further testing in Monterey County, California for five years. This propagation and testing has demonstrated that the combination of traits disclosed herein which characterize the new variety are fixed and retained true to type through successive generations of asexual reproduction.

### SUMMARY OF THE INVENTION

The present invention relates to a new and distinct variety of strawberry named ‘Driscoll Sausalito’. The variety is botanically identified as Fragaria×ananassa. The new variety is distinguished from other varieties by a number of characteristics as set forth in Tables 1-4.

### Comparison to Similar Varieties

The varieties which we believe to be similar to ‘Driscoll Sausalito’ from those known to us are ‘Driscoll Lanai’ (U.S. Plant Pat. No. 15,145) and ‘San Juan’ (U.S. Plant Pat. No. 12,899). There are several characteristics of the new variety that are different from, or not possessed by ‘Driscoll Lanai’ and ‘San Juan’. For example, the new variety has a different internal color, longer petal length, longer flower diameter, different anythocyanin color and coloration and is moderately resistant to powdery mildew.

‘Driscoll Sausalito’ is distinguished from its parent, ‘San Juan’ as indicated in Tables 1-4. Plants of 14C185 were not available for side by side comparison. ‘Driscoll Sausalito’ differs from 14C185 by having brighter red, more uniformly colored berries and superior shelf-life.

### BRIEF DESCRIPTION OF THE DRAWINGS

The accompanying photographs show typical specimens of the new variety, including fruit, foliage and flowers, in color as nearly true as it is reasonably possible to make in color illustrations of these characteristics.

FIG. 1 shows the whole the plant.

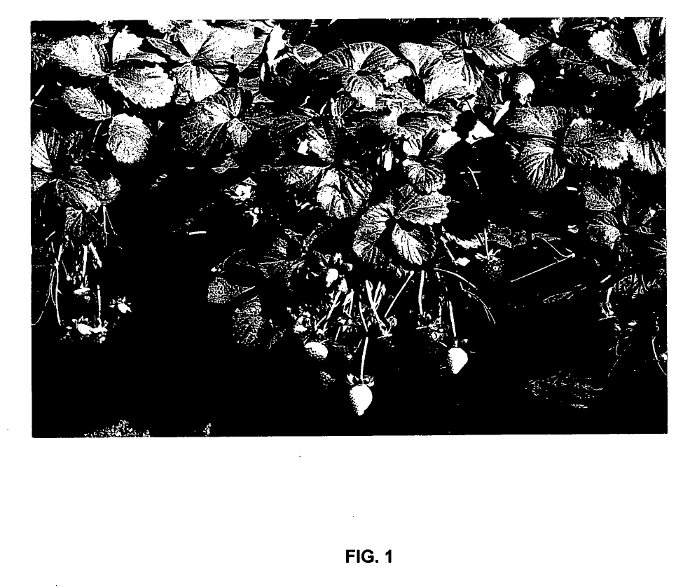

FIG. 2 shows the upper side of the leaves of the plant.

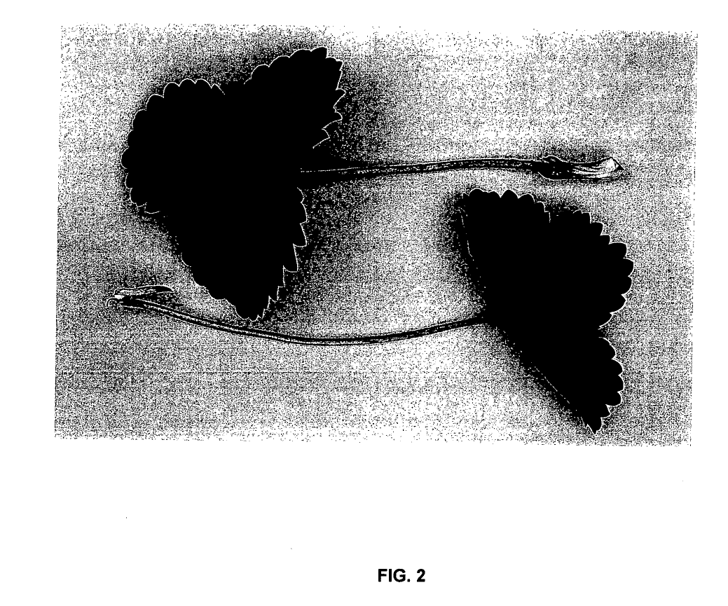

FIG. 3 shows the under side and upper side of the flowers.

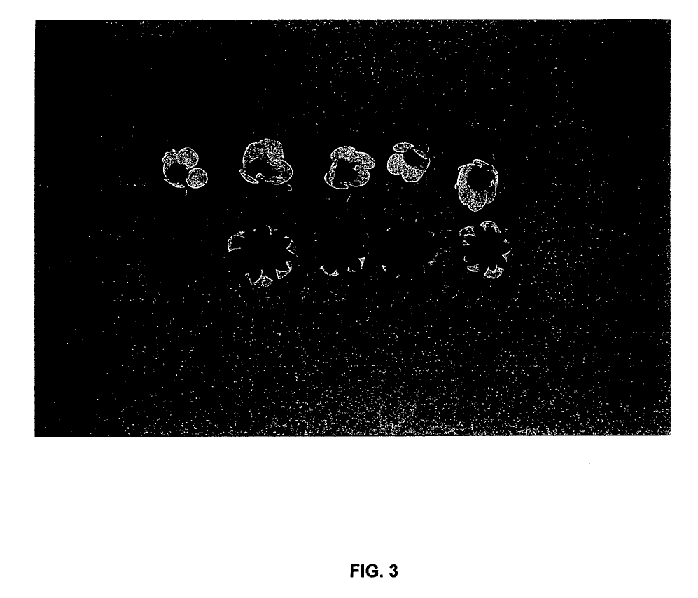

FIG. 4 shows a close-up of the fruit.

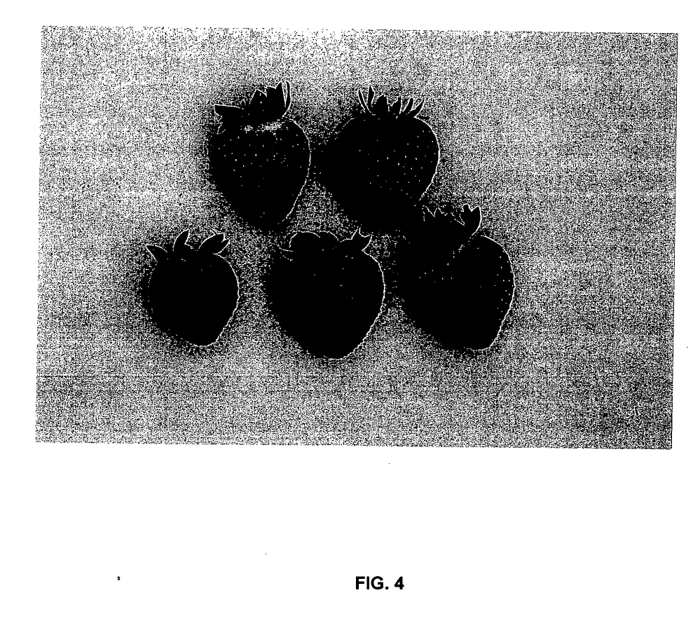

FIG. 5 shows the fruit in longitudinal cross-section.

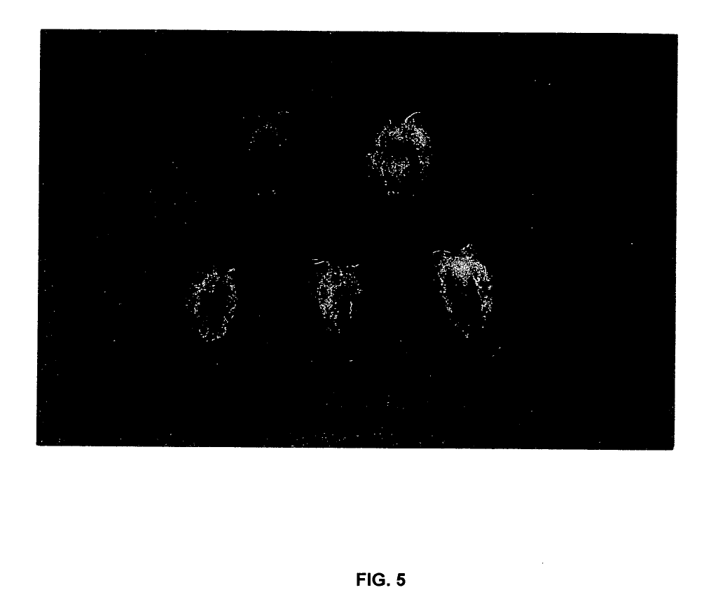

### DESCRIPTION OF THE NEW VARIETY

The following detailed description of the new variety is based upon observations taken of plants harvested from a nursery in Shasta country, California in October, 2004 and held in cold storage until planting in Monterey County, California in November 2004. Plants were grown in raised beds of soil under conditions typical of commercial strawberry production in Monterey County, California. Observations of ‘Driscoll Sausalito’, ‘Driscoll Lanai’, and ‘San Juan’ were made in side by side comparison in July 2005. Fruits were harvested twice weekly from April, 2004 to November, 2004 for yield determination. Color designations, color descriptions, and other phenotypical descriptions may deviate from the stated values and descriptions depending upon variation in environmental, seasonal, climatic and cultural conditions. Colors are described and the most similar color designations are provided from The Royal Horticultural Society (RHS) Colour Chart.

### Propagation

The new variety is principally propagated by way of stolons. Although propagation by stolons is presently preferred, other known methods of propagating strawberry plants may be employed.

### Characteristics of the New Variety

Information on the new variety is presented in Tables 1, 2, 3 and 4. In the tables, the flowers described are secondary flowers except where indicated. The fruit described is the secondary fruit on seven month old plants. Fruit and flower measurements are an average of both primary and secondary fruit and flowers.

Table 1 provides information on the plant and fruit characteristics of the new variety ‘Driscoll Sausalito’ compared with characteristics of ‘Driscoll Lanai’ and ‘San Juan’. Table 2 provides additional information of the plant and fruit characteristics of the new variety ‘Driscoll Sausalito’ compared with characteristics of the varieties ‘Driscoll Lanai’ and ‘San Juan’. Table 3 provides reactions of the new variety to stresses, pests and diseases compared with reactions of the varieties ‘Driscoll Lanai’ and ‘San Juan’. Table 4 provides isozyme characteristics of the new variety as compared to that of the varieties ‘Driscoll Lanai’ and ‘San Juan’.

TABLE 1
QUANTITATIVE COMPARISON OF ‘DRISCOLL SAUSALITO’,
‘DRISCOLL LANAI’ AND ‘SAN JUAN’

| | ‘Driscoll | ‘Driscoll | |
|---|---|---|---|
| | Sausalito’ | Lanai’ | ‘San Juan’ |
| Plant Characteristics | | | |
| Height of Plant (cm) | 24.7 | 23.4 | 28.9 |
| Spread of Plant (cm) | 45.6 | 38.3 | 51.7 |
| Number of Crowns | 3.5 | 3.3 | 4.3 |
| Leaf Characteristics | | | |
| Terminal Leaflet Length | 6.9 | 6.7 | 7.5 |
| (cm) | | | |
| Terminal Leaflet Width | 6.6 | 6.6 | 7.6 |
| (cm) | | | |
| Terminal Leaflet | 1.04 | 1.00 | 0.99 |
| Length/Width Ratio | | | |
| # Teeth/Terminal Leaflet | 17.8 | 21.1 | 20.4 |
| Color of upper side | 137A | 139A | 147A |
| | medium green | medium green | medium to |
| | | | dark green |
| Color of under side | 137C | 138B | 147C |
| | light green | light green | light green |
| Petiole Length (cm) | 19.4 | 18.0 | 25.7 |
| Petiole Diameter (mm) | 3.6 | 2.9 | 3.7 |
| Petiole Color | 145B | 145A | 149A |
| | yellow green | yellow green | yellow green |
| Petiolule Length (mm) | 8.0 | 10.7 | 7.5 |
| Petiolule Diameter (mm) | 1.8 | 1.7 | 2.1 |
| Petiolule Color | 145C | 145A | 149A |
| | yellow green | yellow green | yellow green |
| Bract Frequency | 8% | 0% | 33% |
| | typically | | typically |
| | single | | single |
| Stipule Length (cm) | 3.5 | 3.2 | 3.5 |
| Stipule Width (cm) | 0.9 | 0.8 | 0.7 |
| Stolon Characteristics | | | |
| Anthocyanin color | 185A | 60A | 59A |
| | greyed purple | red purple | red purple |
| Diameter at bract (mm) | 3.16 | 2.93 | 3.49 |
| Avg. # of Daughter | 56 | 59 | 69 |
| plants (2003 Nursery) | | | |
| Flower Characteristics | | | |
| Petal Length (cm) | 1.4 | 1.1 | 1.1 |
| Petal Width (cm) | 1.3 | 1.2 | 1.2 |
| Petal Length/Width Ratio | 1.08 | 0.94 | 0.90 |
| Flower Diameter (cm) | 3.1 | 2.8 | 2.6 |
| Calyx Diameter (cm) | 3.6 | 3.9 | 3.5 |
| Sepal Length (mm) | 11.4 | 11.7 | 11.7 |
| Sepal Width (cm) | 5.5 | 4.8 | 5.6 |
| Petal Color | 155C | 155C | 155C |
| | white | white | white |
| Receptical Color | 154C | 154C | 154C |
| | yellow green | yellow green | yellow green |
| Anther Color | 13A | 9A | 9A |
| | yellow | yellow | yellow |
| Fruiting Truss Length | 25.3 | 25.3 | 33.1 |
| (cm) | | | |
| Fruit Characteristics | | | |
| Fruit Length (cm) | 3.8 | 3.8 | 3.9 |
| Fruit Width (cm) | 3.6 | 4.0 | 4.3 |
| Fruit Length/Width Ratio | 1.04 | 0.96 | 0.90 |
| Average Berry | 2.18 | 23.9 | 25.4 |
| Weight (g) | | | |
| External Color | 46A | 45B | 53A |
| | orange red | orange red | dark red |
| Internal Color | 35A | 47C | 44A |
| | whitish | orange red | red |
| Achene Coloration | 185A to 162B | 185B to 154B | 46B to 16A |
| | greyed purple | greyed purple | dark red to |
| | to greyed | to greyed | yellow |
| | yellow | yellow | |
| Achenes per berry | 195 | 292 | 304 |
| Achene weight(g) | 0.0006 | 0.0005 | 0.0006 |
| 2004 Marketable Yield | 1,283 | 1,377 | 984 |
| (g/plant) | | | |

TABLE 2
QUALITATIVE COMPARISON OF ‘DRISCOLL SAUSALITO’,
‘DRISCOLL LANAI’ AND ‘SAN JUAN’

| | ‘Driscoll | ‘Driscoll | |
|---|---|---|---|
| | Sausalito’ | Lanai’ | ‘San Juan’ |
| Plant | | | |
| Habit | flat | flat | globose to flat |
| | | | globose |
| Canopy Density | medium | open | medium |
| Vigor | weak to | medium | medium |
| | medium | | |
| Leaf | | | |
| Shape in cross section | slightly concave | slightly concave | flat to slightly |
| | | to flat | convex |
| Interveinal blistering | medium | medium | medium to |
| | | | strong |
| Glossiness | weak | weak | weak to |
| | | | medium weak |
| Number of leaflets | three only | three only | sometimes more |
| | | | than 3 leaflets |
| | | | (approx. 17% of |
| | | | leaves) |
| Terminal leaflet margin profile | revolute | revolute | revolute to flat |
| Terminal leaflet shape of base | rounded | rounded | obtuse to |
| | | | rounded |
| Terminal leaflet shape of teeth | rounded | rounded | rounded |
| Stipule pubescence | sparse | medium to | medium |
| | | dense | |
| Petiole pubescence | sparse to | medium dense | medium |
| | medium | | |
| Petiole pose of hairs | outward | downward | outward to |
| | | | downward |
| Stolon | | | |
| Anthocyanin coloration | very strong | strong | strong |
| Thickness | medium | medium to thick | medium |
| Pubescence | very sparse | dense | medium |
| Inflorescence | | | |
| Position relative to foliage | beneath to level | level to above | beneath to level |
| Diameter of calyx relative to | larger | same size to | larger |
| corolla on secondary flowers | | larger | |
| Diameter of inner calyx relative to | larger | larger | same size |
| outer on secondary flowers | | | |
| Spacing of petals | overlapping | overlapping | overlapping |
| Fruiting Truss | | | |
| Attitude at first picking | prostrate | prostrate | prostrate |
| Fruit | | | |
| Predominant shape | conical | conical to ovoid | conical to |
| | | | almost |
| | | | cylindrical |
| Difference in shapes between | slight | slight | moderate |
| primary and secondary fruits | | | |
| Band without achenes | medium | narrow to | narrow |
| | | medium | |
| Unevenness of surface | weak | weak | medium |
| Evenness of color | slightly uneven | even | even |
| Glossiness | strong | strong | very strong |
| Insertion of achenes | level to above | level with | level with |
| | fruit surface | surface | surface |
| Insertion of calyx | level to above | level | level |
| Pose of the calyx segments | spreading to | spreading to | spreading to |
| | reflexed | reflexed | reflexed |
| Size of calyx in relation to fruit on | same size | smaller | same size |
| secondary fruit | | | |
| Adherence of calyx | strong | strong | strong |
| Firmness of flesh | medium | medium | firm |
| Evenness of flesh color | slightly uneven | uneven | slightly uneven |
| | | | to even |
| Distribution of flesh color | marginal and | marginal and | marginal and |
| | central | central | central |
| Hollow center size | small | medium | medium |
| Sweetness | strong | medium | medium to |
| | | | strong |
| Texture when tasted | fine | fine | medium |
| Acidity | weak to | medium | medium |
| | medium | | |
| Time of First Flowering After | mid-February | mid-February | mid-February |
| Planting in 2005 | | | |
| Harvest Interval in 2005 | early April to | late March to | late March to |
| | early November | early November | early November |
| Type of Bearing | partially | partially | partially |
| | everbearing | everbearing | everbearing |

TABLE 3
REACTIONS TO STRESS PESTS AND DISEASES
FOR ‘DRISCOLL SAUSALITO’, ‘DRISCOLL
LANAI’ AND ‘SAN JUAN’

| | ‘Driscoll | ‘Driscoll | |
|---|---|---|---|
| | Sausalito’ | Lanai’ | ‘San Juan’ |
| Reaction to Pests | | | |
| Tetranychus urticae | susceptible | susceptible | moderately |
| | | | susceptible |
| Lygus hesperus | susceptible | susceptible | susceptible |
| Reaction To Diseases | | | |
| Botrytis fruit rot | susceptible | susceptible | susceptible |
| Powdery mildew | moderately | susceptible | susceptible |
| | resistant | | |
| Verticillium wilt | moderately | moderately | susceptible |
| | resistant | resistant | |
| Strawberry Mottle Virus | moderately | moderately | moderately |
| | resistant | resistant | resistant |
| Xanthomonas fragariae | moderately | moderately | moderately |
| | susceptible | susceptible | susceptible |

### Isozyme Analysis

In addition to the morphological description above, the new cultivar ‘Driscoll Sausalito’ has been analyzed to obtain an indication of its genetic makeup to provide further means for identifying the new variety and distinguishing it from other somewhat similar and/or related strawberry varieties. Specifically, leaf samples of ‘Driscoll Sausalito’, ‘Driscoll Lanai’ and ‘San Juan’ were analyzed by electrophoresis for isozyme patterns of the enzymes phosphoglucoisomerase (“PGI”), leucine aminopeptidase (“LAP”) and phosphoglucomutase (“PGM”). See J. Amer. Soc. Hort. Sci. 106:684-687. Isozyme characterization of the three varieties is presented in Table 4, with the letters representing the banding patterns for each enzyme as designated in the above-identified article.

TABLE 4
ISOZYME ANALYSIS FOR ‘DRISCOLL SAUSALITO’,
‘DRISCOLL LANAI’ AND ‘SAN JUAN’

| | ‘Driscoll | ‘Driscoll | |
|---|---|---|---|
| Locus | Sausalito’ | Lanai’ | ‘San Juan’ |
| PGI | A2 | A1 | A2 |
| LAP | B3 | B3 | B3 |
| PGM | C2 | C2 | C4 |

What is claimed:

1. A new and distinct variety of strawberry plant, substantially as shown and described.
